(19) United States
(12) Patent Application Publication
(10) Pub. No.: US 2018/0318247 A1
(43) Pub. Date: Nov. 8, 2018

(54) CATIONIC ANTIMICROBIAL HANDWASH

(71) Applicant: GOJO Industries, Inc., Akron, OH (US)

(72) Inventors: Mitchell Cohen, Salisburg, NC (US); James Bingham, Akron, OH (US)

(21) Appl. No.: 16/037,083

(22) Filed: Jul. 17, 2018

Related U.S. Application Data
(63) Continuation of application No. 14/387,960, filed on Sep. 25, 2014.
(60) Provisional application No. 61/617,917, filed on Mar. 30, 2012.

(51) Int. Cl. A61K 31/23; A61K 8/34; A61K 31/14; A01N 47/44; A61K 8/40; A61K 8/90; A61Q 17/00; A61K 8/60; A61K 8/44; A61K 8/41; A01N 33/12

(52) U.S. Cl. CPC A61K 31/23; Y02A 50/473; A61K 8/34; A61K 31/14; A01N 47/44; A61K 8/345; A01N 2300/00; A61K 8/90; A61Q 17/005; A61K 8/604; A61K 8/44; A61K 8/416; A01N 33/12; A61K 8/40

(57) ABSTRACT

An antimicrobial composition is provided that includes from about 0.1 to about 2 wt. % of a cationic antimicrobial agent selected from the group consisting of lauric arginate and benzalkonium chloride, based upon the total weight of the antimicrobial composition; from about 1 to about 10 wt. % of two or more nonionic surfactants selected from the group consisting of glucoside alkyl ethers and poloxamers, based upon a total weight of the antimicrobial composition; from about 0.1 to about 5 wt. % of an amine oxide, based upon the total weight of the antimicrobial composition; and one or more preservatives selected from phenoxyethanol, ethylhexyl glycerin, hexylene glycol, caprylyl glycol, and mixtures thereof

### CROSS REFERENCE TO RELATED APPLICATIONS

This application is a continuation of, claims priority to, and any other benefit of, U.S. patent application Ser. No. 14/387,960, filed on Sep. 25, 2014, which is National Stage Entry of PCT/US2013/031361, filed on Mar. 14, 2013, which claims priority from U.S. Patent Application Ser. No. 61/617,917. Each of the foregoing are incorporated by reference herein in their entirety.

### FIELD OF THE INVENTION

The present invention relates to an antimicrobial composition, especially a liquid hand soap, that has a proper balance of ingredients for providing a high cleansing ability, high foam, good conditioning and a pleasant emollient effect on the skin.

### BACKGROUND OF THE INVENTION

Handwash compositions are preferably formulated to provide good cleaning, good foaming, and to be mild to the skin. Handwash compositions typically employ a surfactant system (generally containing at least one anionic surfactant and perhaps an additional amphoteric surfactant) to provide cleaning and foaming. Moisturizers or other skin benefit agents may be employed to promote mildness to the skin.

Antimicrobial handwash compositions are formulated to include an antimicrobial agent. However, difficulties are encountered with cationic antimicrobial agents, because the rapid kill associated with cationic actives is inhibited by high levels of surfactants. It is believed that, once the surfactant system reaches its combined critical micelle concentration it begins to pull in the monomers of the cationic surfactant to make mixed micelles. This inactivates the antimicrobial efficacy.

Thus, there remains a need for antimicrobial compositions that provide a balance of effective cleansing, high foam and emollient benefits, without irritating the skin, and without negatively impacting the antimicrobial efficacy of the cationic antimicrobial agent.

### SUMMARY OF THE INVENTION

Embodiments of the present invention provide an antimicrobial handwash comprising from about 0.1 to about 2 wt. % of a cationic antimicrobial agent selected from the group consisting of lauric arginate and benzalkonium chloride, based upon the total weight of the antimicrobial composition; from about 1 to about 10 wt. % of two or more nonionic surfactants selected from the group consisting of glucoside alkyl ethers and poloxamers, based upon a total weight of the antimicrobial composition; from about 0.1 to about 5 wt. % of an amine oxide, based upon the total weight of the antimicrobial composition; and one or more preservatives selected from phenoxyethanol, ethylhexyl glycerin, hexylene glycol, caprylyl glycol, and mixtures thereof.

In one or more embodiments, the foam booster is an amphoteric surfactant. The amount of amphoteric surfactant may be from about 1 to about 5 wt. %, based upon the total weight of the antimicrobial composition. In these or other embodiments, the cationic antimicrobial agent may be benzalkonium chloride, benzethonium chloride, lauric arginate, or a mixture thereof. The amount of the cationic antimicrobial agent may be from about 0.1 to about 2 wt. %, based upon the total weight of the antimicrobial composition. In these or other embodiments, the preservative may be one or more of parabens, phenols, quaternary compounds, C₁₋₆ alcohols, isothiazolones, alkane diols, and alkylene diols. In these or other embodiments, the amount of nonionic surfactant may be from about 1 to about 10 wt. %, based upon a total weight of the antimicrobial composition. In these or other embodiments, the total amount of the surfactant in the antimicrobial composition may be below the critical micelle concentration at standard temperature and pressure.

In one or more embodiments, the cationic antimicrobial handwash comprises from about 0.1 to about 2 wt. %, based upon the total weight of the antimicrobial composition of lauric arginate, from about 1 to about 10 wt. %, based upon a total weight of the antimicrobial composition of decyl glucoside, poloxamer, or a mixture thereof, from about 1 to about 5 wt. %, based upon the total weight of the antimicrobial composition of lauramine oxide, and one or more preservatives selected from phenoxyethanol, ethylhexyl glycerin, hexylene glycol, caprylyl glycol, and mixtures thereof.

In one or more embodiments, the cationic antimicrobial handwash comprises from about 0.1 to about 2 wt. %, based upon the total weight of the antimicrobial composition of benzalkonium chloride, from about 1 to about 10 wt. %, based upon a total weight of the antimicrobial composition of decyl glucoside, poloxamer, or a mixture thereof, from about 1 to about 5 wt. %, based upon the total weight of the antimicrobial composition of lauramine oxide, and one or more preservatives selected from phenoxyethanol, ethylhexyl glycerin, hexylene glycol, caprylyl glycol, and mixtures thereof.

Embodiments of the present invention provide a cationic antimicrobial handwash comprising from about 0.1 to about 2 wt. %, based upon the total weight of the antimicrobial composition of lauric arginate; from about 1 to about 10 wt. %, based upon a total weight of the antimicrobial composition of decyl glucoside, poloxamer, or a mixture thereof; from about 1 to about 5 wt. %, based upon the total weight of the antimicrobial composition of lauramine oxide; and one or more preservatives selected from phenoxyethanol, ethylhexyl glycerin, hexylene glycol, caprylyl glycol, and mixtures thereof.

Embodiments of the present invention provide a cationic antimicrobial handwash comprising from about 0.1 to about 2 wt. %, based upon the total weight of the antimicrobial composition of benzalkonium chloride; from about 1 to about 10 wt. %, based upon a total weight of the antimicrobial composition of decyl glucoside, poloxamer, or a mixture thereof, from about 1 to about 5 wt. %, based upon the total weight of the antimicrobial composition of lauramine oxide; and one or more preservatives selected from phenoxyethanol, ethylhexyl glycerin, hexylene glycol, caprylyl glycol, and mixtures thereof

Advantageously, embodiments of the present invention provide an antimicrobial handwash wherein the rapid broad spectrum antimicrobial efficacy of the cationic antimicrobial agent is not deleteriously affected by the surfactants. Advantageously, embodiments of the present invention provide an antimicrobial handwash wherein the preservative has an enhancing effect on the antimicrobial efficacy of the cationic surfactant. In one or more embodiments, the enhancing effect is unexpectedly synergistic, i.e. is significantly more than just an additive effect.

Embodiments of the present invention further provide an antimicrobial handwash that is useful as a healthcare personnel hand wash according to the standards of the FDA Tentative Final Monograph for Healthcare Antiseptic Drug Products (TFM) (Federal Register 59 [116], Jun. 17, 1994: pp. 31402-31452).

Embodiments of the present invention further provide a hygienic hand wash, as described in one or more of the above embodiments, that is useful as a hygienic hand wash according to standard test method EN 14348:2005.

Embodiments of the present invention further provide a method for killing or inactivating microbes on a surface comprising the steps of applying, to a surface, an effective amount of the antimicrobial composition of any of the preceding embodiments or as further described hereinbelow. In one or more embodiments, the method for killing or inactivating microbes on a surface further comprises the step of rinsing the surface with water after allowing the antimicrobial composition to remain in contact with the surface for a selected period of time.

### DETAILED DESCRIPTION OF THE INVENTION

It has now been discovered that it is possible to formulate an antimicrobial composition, especially a liquid hand soap, that has a proper balance of ingredients for providing a high cleansing ability, high foam, good conditioning and a pleasant emollient effect on the skin, while maintaining the antimicrobial efficacy of the antimicrobial agent.

In one or more embodiments, the antimicrobial composition includes one or more nonionic surfactants, optionally a foam booster, a cationic antimicrobial agent, and a preservative. The nonionic surfactant may provide detergent properties to the antimicrobial liquid hand soap for cleansing the skin. Examples of nonionic surfactants include fatty alcohols such as cetyl alcohol, stearyl alcohol, cetostearyl alcohol, and oleyl alcohol, polyoxyethylene glycol alkyl ethers, such as octaethylene glycol monododecyl ether, and pentaethylene glycol monododecyl ether, polyoxypropylene glycol alkyl ethers, glucoside alkyl ethers, polyoxyethylene glycol octylphenol ethers, polyoxyethylene glycol alkylphenol ethers, such as nonoxynol-9, glycerol alkyl esters such as glyceryl laurate, polyoxyethylene glycol sorbitan alkyl esters, such as polysorbate, sorbitan alkyl esters, cocamide MEA, cocamide DEA, amine oxides, such as dodecyldimethylamine oxide, block copolymers of polyethylene glycol and polypropylene glycol, such as poloxamers, polyethoxylated tallow amine, and mixtures thereof.

In one or more embodiments, the antimicrobial composition includes at least one non-ionic surfactant selected from glucoside alkyl ethers, poloxamers, amine oxides, and combinations thereof. Examples of glucoside alkyl ethers include decyl glucoside, lauryl glucoside, ceteryl glucoside, and octyl glucoside. In one or more embodiments, the glucoside alkyl ether is present in the antimicrobial composition in an amount of from about 1 to about 10 wt. %, in other embodiments, from about 1.5 to about 8 wt. %, and in other embodiments, from about 1.8 to about 7 wt. %, based upon the total weight of the antimicrobial composition.

Examples of poloxamers include triblock polymers. In one or more embodiments, the triblock polymer is a block copolymer based upon polyethylene and polypropylene. Poloxamers are commercially available, for example from BASF under the tradename Pluronic. In one or more embodiments, the triblock polymer is characterized by a molecular mass of about 3600 g/mol, and a percentage of polyoxyethylene content of about 40%. In one or more embodiments, the poloxamer is present in the antimicrobial composition in an amount of from about 1 to about 10 wt. %, in other embodiments, from about 1.5 to about 8 wt. %, and in other embodiments, from about 1.8 to about 7 wt. %, based upon the total weight of the antimicrobial composition.

Examples of amine oxides include dodecyldimethylamine oxide, which is sometimes referred to as lauramine oxide, lauramidopropyl amine oxide, and cocamido propylamine oxide. In one or more embodiments, the amine oxide is present in the antimicrobial composition in an amount of from about 0.1 to about 5 wt. %, in other embodiments, from about 0.2 to about 4 wt. %, and in other embodiments, from about 0.25 to about 3 wt. %, based upon the total weight of the antimicrobial composition.

In one or more embodiments, the antimicrobial composition comprises at least two nonionic surfactants. In one or more embodiments, the antimicrobial composition comprises at least three nonionic surfactants. In one or more embodiments, the antimicrobial composition includes a glucoside alkyl ether, a poloxamer, and an amine oxide. In one or more embodiments, the antimicrobial composition comprises decyl glycoside, a poloxamer, and lauramine oxide.

The amount of nonionic surfactant should be selected such that the total amount of surfactant in the antimicrobial composition is below the critical micelle concentration at standard temperature and pressure.

In one or more embodiments, the total amount of nonionic surfactant is present in a total amount of from about 1 to about 10 weight percent (wt. %), based upon the total weight of the antimicrobial composition. In other embodiments, the amount of nonionic surfactant is from about 2 to about 8 wt. %, in other embodiments, from about 4 to about 6 wt. %, based upon the total weight of the antimicrobial composition.

In one or more embodiments, the optional foam booster is an amphoteric surfactant. Examples of amphoteric surfactants include sultaines, betaines, and mixtures thereof. Examples of sultaines include cocamidopropyl hydroxysultaine. Examples of betaines include cocamidopropyl betaine (also sometimes referred to as coco betaine), lauramidopropyl betaine, cetyl betaine, oleamidopropyl betaine, and ricinoleamidopropyl betaine.

In one or more embodiments, the foam booster is cocamidopropyl hydroxysultaine, cocamidopropyl betaine, lauramidopropyl betaine, cetyl betaine, oleamidopropyl betaine, ricinoleamidopropyl betaine, or a mixture thereof.

The amount of amphoteric surfactant should be selected such that the total amount of surfactant in the antimicrobial composition is below the critical micelle concentration at standard temperature and pressure.

In one or more embodiments, the amount of amphoteric surfactant is from 0 to about 5 wt. %, in other embodiments, about 1 to about 4.5 wt. %, in other embodiments, from about 1.5 to about 4 wt. %, based upon the total weight of the antimicrobial composition.

In one or more embodiments, the cationic antimicrobial agent may comprise benzalkonium chloride, benzethonium chloride, ethyl lauroyl arginate HCl, sometimes referred to as lauric arginate, or mixtures thereof. Ethyl lauroyl arginate HCl is commercially available in a glycerin carrier under the tradename Aminat-G from Vedeqsa, Inc.

In one or more embodiments, the amount of cationic antimicrobial agent is from about 0.01 to about 2 wt. %, in other embodiments, from about 0.05 to about 1 wt. %, based upon the total weight of the antimicrobial composition.

In one or more embodiments, the amount of lauric arginate is from about 0.1 to about 2 wt. %, in other embodiments, from about 0.5 to about 1.5 wt. %, in yet other embodiments, from about 0.6 to about 1 wt. %, based upon the total weight of the antimicrobial composition.

In one or more embodiments, the amount of benzalkonium chloride is from about 0.1 to about 0.2 wt. %, in other embodiments, from about 0.11 to about 0.15 wt. %, based upon the total weight of the antimicrobial composition.

Advantageously, the amount of nonionic surfactant and amphoteric surfactant may be selected to maximize detergency and foam, without negatively influencing the antimicrobial efficacy of the cationic antimicrobial agent. In one or more embodiments, the amount of surfactant relative to the amount of antimicrobial agent may be increased without impeding the antimicrobial efficacy of the antimicrobial agent.

In one or more embodiments, the molar ratio of total surfactant, i.e. nonionic surfactant plus amphoteric surfactant, relative to the amount of antimicrobial agent is from about 5:1 to about 15:1, in other embodiments, from about 5.5:1 to about 12:1, and in yet other embodiments, from about 6:1 to about 10:1.

In one or more embodiments, where the antimicrobial agent is benzalkonium chloride, the molar ratio of total surfactant, i.e. nonionic surfactant plus amphoteric surfactant, relative to the amount of antimicrobial agent is from about 15:1 to about 60:1, in other embodiments, from about 20:1 to about 50:1, and in yet other embodiments, from about 22:1 to about 46:1.

In one or more embodiments, the molar ratio of nonionic surfactant, relative to the amount of antimicrobial agent is from about 5:1 to about 15:1, in other embodiments, from about 5.5:1 to about 12:1, and in yet other embodiments, from about 6:1 to about 10:1.

Optionally, the antimicrobial composition may include one or more preservatives. Examples of suitable preservatives include parabens, phenols, quaternary compounds, C₁₋₆ alcohols such as ethanol, isothiazolones, alkane diols, and alkylene diols. In one or more embodiments, the amount of preservatives is from about 0 to about 2 wt. %, in other embodiments, from about 0.01 to about 1.5 wt. %, in other embodiments, from about 0.05 to about 1 wt. %, based upon the total weight of the antimicrobial composition. Other examples of preservatives include DMDM hydantoin. In one or more embodiments, the amount of preservative is from about 0.3 wt % to about 0.5 wt %, in other embodiments, from about 0.3 wt % to about 0.4 wt %, based upon the total weight of the composition. In one or more embodiment, the preservative includes one or more of phenoxyethanol, ethylhexyl glycerin, hexylene glycol, and caprylyl glycol. In one or more embodiments, the preservative comprises one or more of caprylyl glycol and hexylene glycol. In one or more embodiments, the preservative includes a blend of caprylyl glycol, phenoxyethanol, and hexylene glycol.

Optionally, the antimicrobial composition may include one or more skin benefit agents, such as moisturizers. Examples of moisturizers include urea and urea derivatives such as hydroxyethyl urea. In one or more embodiments, the amount of hydroxyethyl urea is from about 0 to about 20 wt. %, in other embodiments, from about 1 to about 15 wt. %, in yet other embodiments, from about 1.5 to about 12 wt. %.

Optionally, the antimicrobial composition may include one or more chelators. Examples of chelators include ethylenediaminetetraacetic acid (EDTA), and ethylenediamine N,N′-disuccinic acid (EDDS). In one or more embodiments, the amount of chelating agent is from about 0.05 to about 5 wt. %, in other embodiments, from about 0.1 to about 1 wt. %, based upon the total weight of the antimicrobial composition.

The antimicrobial compositions of the present invention may optionally further include one or more additives that are commonly used in soaps. Such additives include thickening agents, fragrance, colorants, and pH adjusters. Examples of pH adjusters include amines such as triethanolamine, inorganic acids, and organic acids such as citric acid.

In one or more embodiments, the above ingredients are added directly to the antimicrobial composition. In other embodiments, one or more of the ingredients are added to the antimicrobial composition as a solution or emulsion. In other words, one or more of the ingredients may be premixed with a carrier to form a solution or emulsion, with the proviso that the carrier does not deleteriously affect the foaming or antimicrobial properties of the antimicrobial composition. Examples of carriers include water, alcohol, glycols such as propylene or ethylene glycol, ketones, linear and/or cyclic hydrocarbons, triglycerides, carbonates, silicones, alkenes, esters such as acetates, benzoates, fatty esters, glyceryl esters, ethers, amides, polyethylene glycols and PEG/PPG copolymers, inorganic salt solutions such as saline, and mixtures thereof It will be understood that, when an ingredient is premixed to form a solution or emulsion, the amount of solution or emulsion that is added to the antimicrobial composition may be selected so that the amount of the ingredient falls within the ranges set forth hereinabove.

The balance of the composition may include water, or other suitable solvent.

In one or more embodiments, the pH of the antimicrobial composition is from about 3 to about 10, in other embodiments, from about 5 to about 7, and in other embodiments, the pH of the antimicrobial composition is from about 5.5 to about 6.5.

The antimicrobial compositions according to the present invention can be prepared in clear or opaque form. Advantageously, they are detersive and also nonirritating to the skin. In one or more embodiments, the antimicrobial compositions are hypoallergenic, have high foam, and good emollient properties.

In one or more embodiments, the antimicrobial composition is effective in killing gram negative and gram positive bacteria, fungi, parasites, non-enveloped and/or enveloped viruses. In one or more embodiments, the antimicrobial composition has rapid antimicrobial efficacy against bacteria such as Staphylococcus aureus, methicillin-resistant S. aureus, Escherichia coli, Pseudomonas aeruginosa, Serratia marcescens, and fungi such as Candida albicans and Aspergillus niger. In one or more embodiments, the antimicrobial composition has rapid efficacy against skin microflora, including resident and transient skin microflora.

Thus, the present invention further provides a method for killing or inactivating microbes on a surface comprising applying, to the surface, an effective amount of an antimicrobial composition as described herein. In one or more embodiments, the antimicrobial composition of the present invention is applied topically to mammalian skin. In one embodiment, the methods of bringing the antimicrobial composition into contact with a microbe on human skin includes applying an amount of the composition to the skin, and allowing the composition to remain in contact with the skin for a suitable amount of time. In other embodiments, the composition may be spread over the surface of the skin, rubbed in, and rinsed off

In one or more embodiments, the present invention includes a method of reducing the amount of bacteria on a surface, such as skin. The method includes contacting the desired surface with an effective amount of a composition according to the present invention, and thereafter, rinsing the hand with water.

It is envisioned that the antimicrobial composition of the present invention may be used as a healthcare personnel hand wash. It is expected that the present invention provides an antimicrobial composition that will meet the standards of the FDA Tentative Final Monograph for Healthcare Antiseptic Drug Products (TFM) (Federal Register 59 [116], Jun. 17, 1994: pp. 31402-31452) for healthcare personnel hand wash.

It is envisioned that the antimicrobial composition of the present invention may be used as a hygienic hand wash when tested according to standard test method EN 14348:2005.

The Examples that follow are intended for illustrating the present invention and not for limiting the scope thereof.

### EXAMPLES

Examples 1-12 were prepared by combining ingredients as summarized in Table 1. The pH of the compositions was 5.0-5.5. The compositions were homogeneous and clear.

In vitro efficacy of these compositions was measured against a mixture of E. coli, S. aureus, E. faecium, and S. marcescens (Group 1). Efficacy was also measured against a mixture of S. aureus (MRSA), P. mirabilis, K pneumoniae, and S. epidermidis (Group 2). The test was conducted according to the ASTM E 2315 method, “Standard Guide for Assessment of Antimicrobial Activity Using a Time-Kill Procedure.” Contact time was 15 seconds. Results are summarized in Table 2 below.

TABLE 1

| INGREDIENT (wt. %) | 1 | 2 | 3 | 4 | 5 | 6 | 7 | 8 | 9 | 10 | 11 | 12 |
|---|---|---|---|---|---|---|---|---|---|---|---|---|
| Water | 92.09 | 92.93 | 96.41 | 93.32 | 94.57 | 90.47 | 89.95 | 89.28 | 84.94 | 84.44 | 88.37 | 95.80 |
| Lauramine Oxide | 0.30 | 0.30 | 0.30 | 0.30 | 0.30 | 0.40 | 0.40 | 0.40 | 0.40 | 0.40 | 0.30 | 0.30 |
| Decyl glycoside | 2.00 | 2.00 | 2.00 | 2.00 | 2.00 | 2.50 | 3.00 | 3.00 | 3.00 | 3.50 | 3.50 | 2.00 |
| Aminat-G | 3.75 | 3.75 | | 2.50 | 1.25 | 3.75 | 3.75 | — | 3.75 | 3.75 | — | — |
| Benzalkonium chloride | — | — | 0.26 | — | — | — | — | 0.26 | — | — | 0.26 | — |
| Poloxamer 124 | 1.00 | 1.00 | 1.00 | 1.00 | 1.00 | 2.00 | 2.00 | 2.00 | 2.00 | 2.00 | 2.00 | 2.50 |
| Barguard CP | 0.85 | — | — | 0.85 | 0.85 | 0.85 | 0.85 | 5.00 | 0.85 | 0.85 | 0.50 | 2.13 |
| Citric Acid | 0.01 | 0.02 | 0.03 | 0.03 | 0.03 | 0.03 | 0.05 | 0.06 | 0.06 | 0.06 | 0.07 | 0.12 |
| Hydroxyethyl urea | — | — | — | — | — | — | — | — | 5.00 | 5.00 | 5.00 | — |

TABLE 2

| | Tier 1 | Tier 2 |
|---|---|---|
| Example No. | Log Kill | Log Kill |
| 1 | >4.92 | >4.81 |
| 2 | 4.5 | 4.7 |
| 3 | 1.2 | >5.0 |
| 4 | >5.0 | 3.3 |
| 5 | >5.0 | 4.7 |
| 6 | 4.6 | 4.5 |
| 7 | >5.0 | >5.0 |
| 8 | 0.6 | 4.7 |
| 9 | 4.6 | 4.5 |
| 10 | >5.0 | >5.0 |
| 11 | 1.2 | >5.0 |
| 12 | >5.0 | 3.8 |

Examples 13-25 were prepared by combining ingredients as summarized in Table 3. The pH of the compositions was 5.0-5.5. The compositions were homogeneous and clear.

In vitro efficacy of these compositions was measured against a mixture of E. coli, S. aureus, E. faecium, and S. marcescens (Group 1). Efficacy was also measured against a mixture of S. aureus (MRSA), P. mirabilis, K. pneumoniae, and S. epidermidis (Group 2). The test was conducted according to the ASTM E 2315 method, “Standard Guide for Assessment of Antimicrobial Activity Using a Time-Kill Procedure.” Contact time was 15 seconds. Results are summarized in Table 4 below.

TABLE 3

| INGREDIENT (wt. %) | 13 | 14 | 15 | 16 | 17 | 18 | 19 | 20 | 21 | 22 | 23 | 24 | 25 |
|---|---|---|---|---|---|---|---|---|---|---|---|---|---|
| Water | 96.23 | 94.19 | 84.19 | 72.16 | 91.86 | 93.15 | 84.19 | 84.17 | 94.18 | 94.25 | 85.31 | 85.90 | 85.41 |
| Lauramine Oxide | — | — | — | — | 0.40 | 0.30 | 1.00 | 1.00 | 1.00 | 1.00 | 0.30 | 0.30 | 0.30 |
| Decyl glycoside | 2.00 | 4.00 | 4.00 | 6.00 | 2.20 | 2.00 | 3.00 | 3.00 | 3.00 | 2.00 | 2.00 | 2.00 | 2.00 |
| Aminat-G | 1.25 | 1.25 | 1.25 | 1.25 | 3.00 | 2.00 | 1.25 | 1.25 | 1.25 | 1.00 | 1.25 | — | 1.25 |
| propylene glycol | — | — | — | — | — | — | — | 10.00 | — | — | — | — | — |
| Poloxamer 124 | — | — | — | — | 2.00 | 2.00 | — | — | — | — | 1.00 | 1.00 | 1.00 |
| Barguard CP | 0.50 | 0.50 | 0.50 | 0.50 | | 0.50 | 0.50 | 0.50 | 0.50 | — | — | — | — |
| Citric Acid | 0.02 | 0.06 | 0.06 | 0.09 | 0.04 | 0.05 | 0.06 | 0.08 | 0.08 | — | 0.045 | 0.05 | 0.04 |
| Hydroxyethyl urea | — | — | 10.00 | 20.00 | — | — | 10.00 | — | — | — | 10.00 | 10.00 | 10.00 |
| phenoxyethanol | — | — | — | — | 0.50 | — | — | — | — | 0.40 | 0.10 | 0.75 | — |
| caprylyl glycol | — | — | — | — | — | — | — | — | — | 0.25 | — | — | — |
| sodium lactate | — | — | — | — | — | — | — | — | — | 0.50 | — | — | — |
| lactic acid | — | — | — | — | — | — | — | — | — | 0.10 | — | — | — |
| EDDS | — | — | — | — | — | — | — | — | — | 0.50 | — | — | — |

TABLE 4

| | Staphylococcus aureus | |
|---|---|---|
| Example No. | Log Kill | Serratia marcescens |
| 13 | 5.4 | 5.4 |
| 14 | 5.4 | 5.4 |
| 15 | 5.4 | 5.4 |
| 16 | 5.4 | 5.4 |
| 17 | 5.4 | 5.4 |
| 18 | 5.4 | 5.4 |
| 19 | 5.4 | 5.4 |
| 20 | 5.4 | 5.4 |
| 21 | 5.4 | 5.4 |
| 22 | >4.3 | >4.3 |
| 23 | 4.3 | >5.5 |
| 24 | 2.5 | 4.4 |
| 25 | 4.2 | >5.5 |

Various modifications and alterations that do not depart from the scope and spirit of this invention will become apparent to those skilled in the art. This invention is not to be duly limited to the illustrative embodiments set forth herein.

What is claimed is:

1. An antimicrobial handwash composition comprising:
from about 0.1 to about 2 wt. % of a cationic antimicrobial agent comprising benzalkonium chloride, based upon the total weight of the antimicrobial composition;
from about 1 to about 10 wt. % of two or more nonionic surfactants selected from the group consisting of glucoside alkyl ethers and poloxamers, based upon a total weight of the antimicrobial composition;
from about 0.1 to about 5 wt. % of an amine oxide, based upon the total weight of the antimicrobial composition; and
one or more preservatives selected from the group consisting of phenoxyethanol, ethylhexyl glycerin, hexylene glycol, caprylyl glycol, and mixtures thereof.

2. The composition of claim 1, wherein a first nonionic surfactant is selected from the group consisting of decyl glucoside, lauryl glucoside, and ceteryl glucoside, and a second nonionic surfactant is selected from the group consisting of lauramine oxide, lauramidopropyl amine oxide, cocamido propylamine oxide, triblock copolymers of polyethylene glycol and polypropylene glycol, and mixtures thereof.

3. The composition of claim 1, wherein the amount of the surfactant in the antimicrobial composition is below the critical micelle concentration at standard temperature and pressure.

4. The composition of claim 1, wherein the total amount of nonionic surfactants present in the composition is from about 1 to about 10 wt. %, based upon a total weight of the antimicrobial composition.

5. The composition of claim 1, further comprising a foam booster.

6. The composition of claim 5, wherein the foam booster is an amphoteric surfactant.

7. The composition of claim 6, wherein the amphoteric surfactant is selected from the group consisting of sultaines, betaines, amine oxide, and mixtures thereof.

8. The composition of claim 7, wherein the amphoteric surfactant is selected from the group consisting of cocamidopropyl hydroxysultaine, cocamidopropyl betaine, lauramidopropyl betaine, cetyl betaine, oleamidopropyl betaine, and ricinoleamidopropyl betaine.

9. The composition of claim 6, wherein the amount of amphoteric surfactant is from about 1 to about 5 wt. %, based upon the total weight of the antimicrobial composition.

10. The composition of claim 1, wherein the composition comprises from about 0.1 to about 0.2 wt. % of benzalkonium chloride, based upon the total weight of the antimicrobial composition.

11. The composition of claim 1, wherein the molar ratio of nonionic surfactant, relative to the amount of antimicrobial agent, is from about 5:1 to about 15:1.

12. The composition of claim 1, wherein the molar ratio of total surfactant, relative to the amount of benzalkonium chloride, is from about 15:1 to about 60:1.

13. The composition of claim 1, wherein the one or more preservatives is selected from the group consisting of parabens, phenols, quaternary compounds, C₁₋₆ alcohols, isothiazolones, alkane diols, and alkylene diols.

14. The composition of claim 13, wherein the one or more preservatives is selected from the group consisting of phenoxyethanol, ethylhexyl glycerin, hexylene glycol, caprylyl glycol, or mixtures thereof.

15. The composition of claim 1, wherein the composition comprises from about 0.01 to about 1.5 wt. % of the one or more preservatives, based upon the total weight of the antimicrobial composition.

16. The composition of claim 1, wherein the one or more preservatives consists of a blend of caprylyl glycol, phenoxyethanol, and hexylene glycol.

17. The composition of any of claim 1, wherein the pH of the composition is from about 5.5 to about 6.5.

18. An antimicrobial composition comprising:
from about 0.1 to about 2 wt. % of benzalkonium chloride, based upon the total weight of the antimicrobial composition;
from about 1 to about 10 wt. % of two or more nonionic surfactants selected from the group consisting of glucoside alkyl ethers and poloxamers, based upon a total weight of the antimicrobial composition;
from about 0.1 to about 5 wt. % of lauramine oxide, based upon the total weight of the antimicrobial composition; and
one or more preservatives selected from the group consisting of phenoxyethanol, ethylhexyl glycerin, hexylene glycol, caprylyl glycol, and mixtures thereof.

19. An antimicrobial composition comprising:
from about 0.1 to about 2 wt. % of benzalkonium chloride, based upon the total weight of the antimicrobial composition;
one or more glucoside alkyl ethers;
one or more poloxamers
an amine oxide; and
one or more preservatives selected from the group consisting of phenoxyethanol, ethylhexyl glycerin, hexylene glycol, caprylyl glycol, and mixtures thereof.

20. The composition of claim 19, wherein the glucoside alkyl ether consists of decyl glycoside, and the amine oxide consists of lauramine oxide.
